(19) United States
(12) Patent Application Publication
(10) Pub. No.: US 2018/0185052 A1
(43) Pub. Date: Jul. 5, 2018

(54) OSCILLATING DRIVE ULTRASONIC SURGICAL DEVICE AND METHODS OF USE

(71) Applicants: Zhao ying Zhou, Beijing (CN); Yu li Zhang, Beijing (CN); Xiao ning Luo, Beijing (CN); Qing Zhou, Beijing (CN); Wei Tian, Beijing (CN)

(72) Inventors: Zhao ying Zhou, Beijing (CN); Yu li Zhang, Beijing (CN); Xiao ning Luo, Beijing (CN); Qing Zhou, Beijing (CN); Wei Tian, Beijing (CN)

(21) Appl. No.: 15/693,246

(22) Filed: Aug. 31, 2017

(30) Foreign Application Priority Data
Aug. 31, 2016 (CN) 201621033874.8
Aug. 31, 2016 (CN) 201621034195.2

(51) Int. Cl. A61B 17/32; A61B 17/16

(52) U.S. Cl. CPC A61B 17/320068; A61B 17/1631; A61B 17/1628; A61B 17/1626

(57) ABSTRACT

Ultrasonic surgical device is provided. The device includes a shell which holds an energy converter and an amplitude-change pole. The energy converter may include piezoceramic stack that converts electrical signals into mechanical ultrasonic movement in the longitudinal direction of the device. The amplitude-change pole is a mechanical amplifier that uses mass differentials to amplify the ultrasonic motion. A cutting instrument interfaces with the amplitude-change pole. A drive motor causes on oscillation mechanism, which may be a rocker, to oscillate in a direction tangentially to the longitudinal axis. The oscillating mechanism may then couple to the energy converter, causing the entire cutting structure to move in this oscillation. This causes the cutter to ultrasonically vibrate along the longitudinal axis, while oscillating tangentially to the longitudinal axis.

### CROSS REFERENCE TO RELATED APPLICATION

This application claims the benefit of Chinese application entitled “The ultrasonic handle and the ultrasonic surgical system, which are used in the ultrasonic surgical system”, Chinese application Ser. No. 201621034195.2, filed in the SIPO on Aug. 31, 2016, all of which is incorporated herein by reference.

This application also claims the benefit of Chinese application entitled “The ultrasonic handle and the ultrasonic surgical system, which are used in the ultrasonic surgical system”, Chinese application Ser. No. 201621033874.8, filed in the SIPO on Aug. 31, 2016, all of which is incorporated herein by reference.

The present invention is related to commonly owned U.S. application Ser. No. 15/693,281 filed on Aug. 31, 2017 in the USPTO, entitled “Rotary Drive Ultrasonic Surgical Device and Methods of Use,” the contents of which are incorporated herein by reference.

### BACKGROUND

The present invention relates generally to surgical systems, and specifically to oscillating driven ultrasonic surgical devices for cutting bone.

During many surgical situations it may become necessary to cut through bone structures in order to replace faulty orthopedic elements or to access shielded anatomy of the patient. Historically, manual saws have been utilized for these bone cutting processes. More recently, motorized reciprocating saws have been utilized. While saws are effective at cutting through the bone, the saw can result in splintering or rough bone edging which may complicate healing times. When fine tooth saws are utilized, the risk of splintering decreases, and the edges may be made smoother, however significant heat can be generated during the cutting process. Further, regardless of the saw type and tooth spacing, all saws have sharp edges that have the potential to injure surrounding tissue.

In addition to saws, scissoring style cutters may be utilized, such as costotomes, may be employed. These cutters also have the potential to splinter the bone, and may induce crushing damage if not kept sharp and in good condition. Further, these cutters are often limited to use in particular regions of the body or on bone structures of particular diameter.

Recently, sonic cutters have been utilized for bone cutting. Ultrasonic cutters feature precision cutting, higher safety, tissue selectivity and low-temperature operation in a way to increase the ease of use for a surgeon, improve the quality of the surgical operation and reduce/relieve pain in the patients. However, traditional ultrasonic bone cutters are limited in operation by only allowing back-and-forth vibration in a vertical orientation which is not efficient when cutting bone structures. Due to the low efficiency, friction between the incision/wound site is exacerbated resulting in excessive damage to the surrounding tissue. Further, the temperature of cutting surface is raised, which may further compound the problem by resulting in thermal injury of nerves and blood vessels around the wound.

It is therefore apparent that an urgent need exists for improved ultrasonic bone cutting devices that effectively cut the bone tissue, minimize adjacent wound damage, and are easily employed by a surgeon.

### SUMMARY

To achieve the foregoing and in accordance with the present invention, an oscillating drive ultrasonic surgical device is provided.

In some embodiments, the ultrasonic surgical device includes a shell which holds an energy converter and an amplitude-change pole. The energy converter may include piezoceramic stack that converts electrical signals into mechanical ultrasonic movement in the longitudinal direction of the device. The amplitude-change pole is a mechanical amplifier that uses mass differentials to amplify the ultrasonic motion. A cutting instrument interfaces with the amplitude-change pole.

The system also includes an oscillating drive device comprising an oscillating mechanism and drive motor. The motor causes the mechanism, which may be a rocker, to oscillate in a direction tangentially to the longitudinal axis. The oscillating mechanism may then couple to the energy converter, causing the entire cutting structure to move in this oscillation. This causes the cutter to ultrasonically vibrate along the longitudinal axis, while oscillating tangentially to the longitudinal axis.

The oscillation may move between 1-40 degrees, 1-30 degrees and 1-20 degrees. Smaller oscillation angles provides for more device control, while trading off the cutting efficiency. As such, in some cases the user may be enabled to alter the oscillation angle of the device.

Note that the various features of the present invention described above may be practiced alone or in combination. These and other features of the present invention will be described in more detail below in the detailed description of the invention and in conjunction with the following figures.

### BRIEF DESCRIPTION OF THE DRAWINGS

In order that the present invention may be more clearly ascertained, some embodiments will now be described, by way of example, with reference to the accompanying drawing, in which:

FIG. 1 shows a diagram of an oscillating drive ultrasonic surgical device.

### DETAILED DESCRIPTION

The present invention will now be described in detail with reference to several embodiments thereof as illustrated in the accompanying drawings. In the following description, numerous specific details are set forth in order to provide a thorough understanding of embodiments of the present invention. It will be apparent, however, to one skilled in the art, that embodiments may be practiced without some or all of these specific details. In other instances, well known process steps and/or structures have not been described in detail in order to not unnecessarily obscure the present invention. The features and advantages of embodiments may be better understood with reference to the drawings and discussions that follow.

Aspects, features and advantages of exemplary embodiments of the present invention will become better understood with regard to the following description in connection with the accompanying drawing(s). It should be apparent to those skilled in the art that the described embodiments of the present invention provided herein are illustrative only and not limiting, having been presented by way of example only. All features disclosed in this description may be replaced by alternative features serving the same or similar purpose, unless expressly stated otherwise. Therefore, numerous other embodiments of the modifications thereof are contemplated as falling within the scope of the present invention as defined herein and equivalents thereto. Hence, use of absolute and/or sequential terms, such as, for example, “will,” “will not,” “shall,” “shall not,” “must,” “must not,” “first,” “initially,” “next,” “subsequently,” “before,” “after,” “lastly,” and “finally,” are not meant to limit the scope of the present invention as the embodiments disclosed herein are merely exemplary.

Further, terminologies, such as “Installation”, “Linkage”, “Connection”, “Fixation”, should be understood in a broad sense unless there exists addition specifications and limitations. For example, it could be permanent connection, detachable connection or all-in-one, electric connection or intercommunication, direct connection or indirect connection through intermediation, it could also be the inner connection or the interconnection of the two components. As to the ordinary technical personnel who could analyze the implications of the above terminologies in the utility model according to the specific circumstances.

The present invention relates to an ultrasonic surgical device. As noted previously, current ultrasonic systems operate by vibrating the cutting instrument in a vertical orientation in a back-and-forth manner. This cutting motion causes low-efficiencies when cutting through bone structures. Low efficiency cuts have a number of drawbacks: they increase friction at the wound site, and result in increased temperature at the point of cutting. The friction on the incision site may result in direct damage to surrounding tissue. Damage may cause later complications, longer healing time, and greater post-operative pain for the patient. The added heat at the cutting location may force the surgeon to cut for shorter periods of time to avoid undue thermal damage to the surrounding tissue. This may increase the difficulty of use for the surgeon. Further even when taking frequent breaks to allow the cut location and tool to cool, small vessels and nerves proximate to the cutting location will be damaged by the excess heat regardless.

Lastly, the inefficient cutting of these traditional systems simply prolongs the cutting process. For larger bone structures this may cause fatigue for the surgeon. Further, the longer a patient remains in surgery, generally the lower the expected outcome, and increased surgical cost. As such, there is incentive to shorten surgical procedures.

The presently described ultrasonic surgical device improves on these older systems by allowing angled oscillation of the cutting instrument. This angled oscillation enables more efficient cutting of bone structures, thereby reducing friction on surrounding tissues, reduced heat generation and shorter overall cutting times. The present system also addresses drawbacks related to osteotomy grinding and fatigue caused to the surgeon due to the strong vibrational forces caused by traditional ultrasonic cutters.

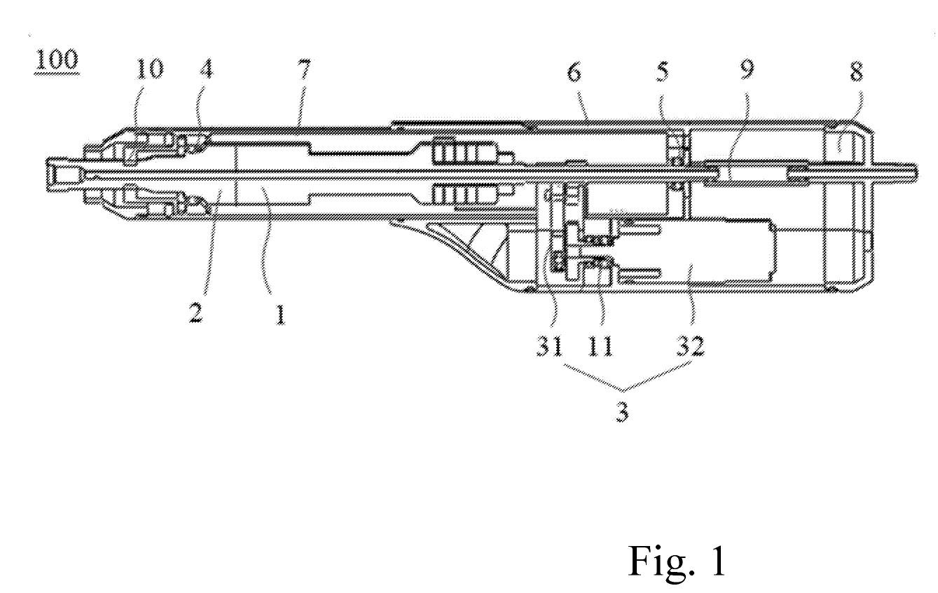

To facilitate discussion, attention is drawn to FIG. 1, which provides a diagram of the ultrasonic surgical device handle, as seen generally at 100. In this example illustration, the ultrasonic device handle is seen as comprising an outer shell, an energy converter 1, amplitude-change pole 2, ultrasonic bone cutter (installed in the front side of amplitude-change pole 2, not illustrated in the FIGURE) and oscillating drive device 3. The energy converter 1, is set in the shell, which is used to convert the ultrasonic signal into a mechanical wave. As with many traditional ultrasonic devices, the energy converter 1 may include a piezoceramic stack that converts the electrical signals into the ultrasonic vibrations.

Similarly, the amplitude-change pole 2 is set in the shell. Specifically, the amplitude-change pole 2 is connected between the ultrasonic bone cutter and energy converter 1. The amplitude-change pole 2 in some embodiments may include a mechanical amplifier that adjusts the amplitude of the cutting instrument based on the configuration of masses on each end of the structure. The amplitude-change pole 2 magnifies the amplitude of the mechanical wave of energy converter 1, and then delivers the mechanical wave to the ultrasonic bone cutter in a manner that causes the ultrasonic bone cutter to vibrate in a vertical direction. The term “vertical” in this context is in relation to the longitudinal axial orientation of the cutting device.

The oscillating drive device 3 includes an oscillating mechanism 31 and a drive motor 32. The drive motor 32 provides an oscillating force that enables the oscillating mechanism 31, amplitude-change pole 2, and energy converter 1 to oscillate in a tangential direction from the central axis of the ultrasonic bone cutter. This movement is perpendicular to the vibrational vertical movement produced by the energy converter 1. This rotational action may have an oscillating angle is from 1° to 40°. Put another way, the angle of the cutting instrument may vary by 20° above and 20° degrees below the device's longitudinal axis.

In some embodiment, the oscillating mechanism 31 may be the crank-rocker mechanism, but not restricted to it. Specifically, the drive motor 32 creates a rotational motion, which moves the oscillating drive device 31 up-and-down. The oscillating mechanism 31 changes the rotational motion to oscillation and mobilizes the energy converter 1. The energy converter 1 then delivers the oscillation to the ultrasonic bone cutter through the amplitude-change pole 2 so as to realize a circumstantial vibration of the ultrasonic bone cutter.

The ultrasonic handle 100 may be used in a larger ultrasonic surgical system by coupling it to the cutting instrument, an energy supply, and a display system that allows configuration of the ultrasonic power levels, and in some cases the angle of oscillation. In this system, the drive motor 32, drives the oscillating mechanism 31 to generate an oscillating motion. This motion is supplied to the drive energy converter 1. The bone cutter instrument installed in the front of the ultrasonic handle 100 has the ability to oscillate vertically and realize the circumstantial oscillation at the same time by virtue of the movement of the oscillating mechanism 31 rocking action. This allows the ultrasonic bone cutter to create the compound motion with longitudinal vibration and circumferential oscillation.

A larger ultrasonic surgical system may include, in addition to the dedicated ultrasonic bone cutter, additional instruments to allow for incision, drilling and grinding in the bone structure, which not only simplifies the surgical process through enhancing the cutting efficiency, but also reduces the friction between ultrasonic bone cutter and wound surface in a way to decrease the cutting temperature of the wound surface, and avoid bone breakage (splintering and rough edging) due to the stress concentration of ultrasonic bone cutter. Furthermore the present system allows the operator to hold the ultrasonic handle conveniently and obtain the perfect efficiency in the aspects of cutting and grinding the bone through by having the oscillating angle from 1° to 40° and modifying the vibration of the ultrasonic handle 100. In some cases it may also be possible to modify the oscillation angle, thereby allowing the operator to more accurately perform the incision and grinding in the bone.

For example, in some systems the oscillating angle is from 1° to 30°, which improves the high-efficient cutting and grinding in bone compared to traditional systems, but reduces the vibration allowing for more detailed operation. In other embodiments, the oscillating angle is from 1° to 20°, which could while slightly less efficient than a larger angle of oscillation still is a significant improvement over traditional ultrasonic cutters, and allows even less vibration therefore improving a firm grip for the operator.

In some embodiments, the size of the oscillating mechanism 31 rocker may be varied to increase or decrease the angle of oscillation of the system. For example, a larger rocker will result in larger longitudinal motion and hence a larger angle of oscillation. In alternate embodiments, the oscillating mechanism 31 may include a configurable gearing that allows for operator configuration of the angle of oscillation.

The energy converter 1 is coaxially connected with the amplitude-change pole 2. The amplitude-change pole 2 is supported by a bearing 4 of the amplitude-change pole, and the energy converter 1 is supported by the bearing 5 of energy converter. The energy converter 1 generates the vertical ultrasonic motion. Simultaneously, the energy converter 1 and the amplitude-change pole 2 are able to vibrate in the circumferential direction, as driven by the oscillating mechanism 31.

The shell may contain the main body of shell 6, sleeve 7 and tail-hood 8. In some embodiments, the main body of shell 6 holds the energy converter 1, oscillating mechanism 31 and drive motor 32. A portion of the sleeve 7 extends into and couples with the main body of the shell 6. The sleeve 7 is used to hold the amplitude-change pole 2. The tail-hood 8 is located distally from the sleeve 7, and seals the main body 6 of the shell in a way to prevent dust and sundries from entering into the inside of the ultrasonic handle 100.

The ultrasonic handle 100 used in the ultrasonic system also includes the flexible connecting tube 9, the interior side of which may be connected with energy converter 1, and the outer side of which is connected with tail-hood 8. The flexible connecting tube 9 belongs to a hollow structure with a liquid pathway to allow the administration of fluid to the cutting site. The fluid may include saline solution, which may enter into the operation region through the liquid pathway of the flexible connecting tube 9.

The side of the sleeve 7 used to install the ultrasonic bone cutter instrument is distally located away from the tail-hood 8. This side is sealed by a flexible gasket 10. In some embodiments, the flexible gasket 10 seals and protects the ultrasonic handle 100 from intrusion of biological materials or other debris. FIG. 1 illustrates that the inner ring of the flexible gasket 10 is fixed in the amplitude-change pole 2, and its outer ring is fixed in the sleeve 7. The flexible gasket 10 is elastic enough to accomplish the sealing of the sleeve 7 while still allowing the amplitude-change pole 2 to vibrate.

In the specific example displayed in FIG. 1, the drive motor 32 is located locates the underneath (inferior to) the energy converter 1. A motor shaft of the drive motor 32 is supported by a bearing 11 of the motor shaft.

While this invention has been described in terms of several embodiments, there are alterations, modifications, permutations, and substitute equivalents, which fall within the scope of this invention. Although sub-section titles have been provided to aid in the description of the invention, these titles are merely illustrative and are not intended to limit the scope of the present invention. It should also be noted that there are many alternative ways of implementing the methods and apparatuses of the present invention. It is therefore intended that the following appended claims be interpreted as including all such alterations, modifications, permutations, and substitute equivalents as fall within the true spirit and scope of the present invention.

What is claimed is:

1. An ultrasonic surgical device comprising:
a shell;
an energy converter contained within the shell;
an ultrasonic bone cutting instrument;
an amplitude-change pole contained within the shell and located between the ultrasonic bone cutting instrument and the energy converter; and
an oscillating drive device comprising an oscillating mechanism and drive motor, wherein the drive motor causes the oscillating mechanism to oscillate the ultrasonic bone cutting instrument along the central axis of the ultrasonic bone cutting instrument, and further wherein the oscillating has an oscillating angle of 1° to 40°.

2. The ultrasonic surgical device of claim 1, further comprising a handle enabling configuration of the oscillating angle.

3. The ultrasonic surgical device of claim 1, wherein the oscillating angle is between 1° to 30°.

4. The ultrasonic surgical device of claim 1, wherein the oscillating angle is between 1° to 20°.

5. The ultrasonic surgical device of claim 2, wherein the energy converter is coaxially connected to the amplitude-change pole.

6. The ultrasonic surgical device of claim 5, wherein the energy converter is held within the handle by an energy converter bearing, and the amplitude-change pole is held within the handle by an amplitude-change pole bearing.

7. The ultrasonic surgical device of claim 2, wherein the shell further comprises a main body, a sleeve and a tail-hood, wherein a top portion of the sleeve extends into and reversibly couples to the main body, and the tail-hood couples to the main body and seals the main body.

8. The ultrasonic surgical device of claim 7, wherein the handle is coupled to a flexible tube, wherein the interior side of the flexible tube couples to the energy converter and the exterior side of the flexible tube couples to the tail-hood.

9. The ultrasonic surgical device of claim 7, further comprising a flexible gasket for sealing a bottom portion of the sleeve, wherein the bottom portion is distally located to the top portion.

10. The ultrasonic surgical device of claim 1, wherein the drive motor is located proximately and inferior to the energy converter.

11. The ultrasonic surgical device of claim 10, further comprising a drive motor shaft extending from the drive motor, wherein the drive motor shaft is supported by a drive motor shaft bearing.

12. An ultrasonic surgical system comprising:
an ultrasonic surgical device as presented in claim 1;
a power supply; and
a display system configured to display the angle of oscillation of the ultrasonic surgical device.

13. A method of treating target tissue within a body using an ultrasonic surgical device according to claim 1, wherein the method includes oscillating an ultrasonic bone cutting instrument and physically contacting the ultrasonic bone cutting instrument the target tissue, by an operator, to induce cutting, wherein an angle of oscillation for the ultrasonic surgical device is configured by the operator of the device.

14. The method of claim 13, wherein the target tissue is a bone structure.
